(19) United States
(12) Patent Application Publication
(10) Pub. No.: US 2026/0004423 A1
(43) Pub. Date: Jan. 1, 2026

(54) Method, System and Device for Performing Measurement and Classification for CT-FFR, and Storage Medium

(71) Applicant: Siemens Healthineers AG, Forchheim (DE)

(72) Inventor: Jing Yan, Shanghai (CN)

(73) Assignee: Siemens Healthineers AG, Forchheim (DE)

(21) Appl. No.: 19/250,368

(22) Filed: Jun. 26, 2025

(30) Foreign Application Priority Data
Jun. 27, 2024 (CN) 202410852486.5

(51) Int. Cl. G06T 7/00; A61B 6/00; A61B 6/03; A61B 6/50; G06T 7/60; G06V 10/764

(52) U.S. Cl. CPC G06T 7/0012; A61B 6/032; A61B 6/504; A61B 6/507; A61B 6/5217; G06T 7/60; G06V 10/764; G06T 2207/10081; G06T 2207/30096; G06T 2207/30104; G06T 2207/30172; G06V 2201/03

(57) ABSTRACT

A method, system and device for performing measurement and classification for CT-FFR, and a storage medium, for dividing lesions into different types according to a Coronary Computed Tomography Angiography (CCTA) image, determining focus lengths of lesion positions according to different lesion types, then determining Computed Tomography Fractional Flow Reserve measurement ranges according to the focus lengths of different lesion types, establishing statistical matrices to subject all Computed Tomography Fractional Flow Reserve (CT-FFR) values within the measurement ranges to calculation, and finally subjecting the calculation results to hemodynamical classification. By performing lesion specific classification of lesions in a hemodynamical sense, the accuracy and stability of CT-FFR assessment is enhanced, interference is reduced, and a more reliable solution is provided for clinical physicians and patients.

### CROSS-REFERENCE TO RELATED APPLICATIONS

The present application claims priority to and the benefit of China patent application no. CN 202410852486.5, filed on Jun. 27, 2024, the contents of which are incorporated herein by reference in their entirety.

### TECHNICAL FIELD

The present disclosure relates to a method, system, and device for performing measurement and classification for CT-FFR, and a storage medium.

### BACKGROUND

CT-FFR (Computed Tomography Fractional Flow Reserve), is a non-invasive technique for assessing functional stenosis in coronary artery disease, and has good correlation with invasive FFR (Fractional Flow Reserve) in terms of diagnostic performance, but nonetheless has certain drawbacks. Firstly, manually selected measurement positions may introduce subjectivity, affecting the uniformity of results. Secondly, CT-FFR values within the range of 2 cm-3 cm distal to the stenosis might vary due to differences in centerline points and lumen contour, resulting in uncertainty of measurement results. In addition, when the CT-FFR value lies in a “gray zone” between 0.75 and 0.8, it is of little diagnostic value, and might lead to misjudgment.

The statement here merely provides background art related to the present disclosure, and does not necessarily form prior art.

### SUMMARY

An object of the present disclosure is to provide a method, system, and device for performing measurement and classification for CT-FFR, and a storage medium, which improve measurement accuracy, enhance the accuracy and stability of assessment, reduce interference, and provide a more reliable solution for clinical physicians and patients.

To achieve the above objects, the present disclosure provides a method for performing measurement and classification for Computed Tomography Fractional Flow Reserve, comprising:
dividing lesions into different lesion types, according to branch involvement and calcification and length of lesions displayed in a Coronary Computed Tomography Angiography (CCTA) image;
determining focus lengths of lesion positions according to the different lesion types;
determining measurement ranges for Computed Tomography Fractional Flow Reserve according to the focus lengths of the different lesion types;
obtaining all Computed Tomography Fractional Flow Reserve (CT-FFR) values within the measurement ranges, subjecting all the CT-FFR values to statistical calculation, and performing lesion specific classification of calculation results according to cut-off values from hemodynamical classification, to obtain a lesion specific classification result.

A lesion type classification function is provided in this solution, being able to identify lesions of different types, select a suitable focus length measurement strategy according to the different lesion types, and thereby determine measurement ranges for CT-FFR values of specific lesions so as to increase the accuracy of measurement. By performing lesion specific classification of lesions in a hemodynamical sense, the accuracy and stability of CT-FFR assessment is enhanced, interference is reduced, and a more reliable solution is provided for clinical physicians and patients.

Optionally, the method of automatically dividing lesion types comprises:
if the length of a lesion is less than or equal to 10 mm, with mild or no calcification, and no principal branch involvement, then it is determined as being a single focal lesion;
if the length of a lesion is greater than or equal to 20 mm, with principal branch involvement, and excessive proximal twisting, then it is determined as being a diffuse lesion;
if the length of an entire lesion is more than 10 mm but less than 20 mm, with moderate or severe calcification, and an irregular contour, then it is determined as being a serial lesion; if lesions are spaced apart by less than 10 mm, then they are determined as being a continuous serial lesion, and if lesions are spaced apart by 10 mm or more, then they are determined as being a discontinuous serial lesion.

In this solution, by identifying lesions of different types such as single focal lesions, diffuse lesions and serial lesions, a suitable focus length measurement strategy can be chosen according to different lesion types, and measurement ranges for CT-FFR values of specific lesions can be determined so as to increase the accuracy of measurement.

Optionally, the method of determining focus lengths of lesion positions comprises:
for a single focal lesion, separately recording a lesion proximal centerline point LPₚᵣₒ and a lesion distal centerline point LP_{dis};
for a diffuse lesion, separately recording a lesion proximal centerline point LPₚᵣₒ and a lesion distal centerline point LP_{dis};
for a serial lesion, first determining a proximal centerline point LPₚᵣₒ and a lesion distal centerline point LP_{dis} of the entire lesion, then determining continuity of the entire lesion, to determine whether the serial lesion is a continuous serial lesion or a discontinuous serial lesion;
for a continuous serial lesion, separately recording a lesion proximal centerline point LPₚᵣₒ and a lesion distal centerline point LP_{dis};
for a discontinuous serial lesion, first recording a lesion proximal centerline point LPₚᵣₒ and a lesion distal centerline point LP_{dis} of the entire lesion, then separately recording a lesion proximal centerline point LPₚᵣₒ′ and a lesion distal centerline point LP_{dis}′ of each lesion.

In this solution, based on the different lesion types, a matching method is chosen to determine the lesion proximal centerline point and lesion distal centerline point, so as to determine measurement ranges for CT-FFR values of specific lesions according to different focus lengths, in order to increase measurement accuracy.

Optionally, for single focal lesions, diffuse lesions and continuous serial lesions, the method of determining the measurement range for Computed Tomography Fractional Flow Reserve comprises:
calculating a centerline point MP_{beg} for the start of measurement and a centerline point MP_{end} for the end of measurement:

$${MP}_{beg} = {LP}_{dis} + 2 cm$$

wherein the centerline point MP_{beg} for the start of measurement is 2 cm longitudinally along the coronary artery from the lesion distal centerline point LP_{dis} in the lesion distal direction;

$${MP}_{end} = {LP}_{dis} + 3 cm$$

wherein the centerline point MP_{end} for the end of measurement is 3 cm longitudinally along the coronary artery from the lesion distal centerline point LP_{dis} in the lesion distal direction.

In this solution, the centerline point MP_{beg} for the start of measurement and the centerline point MP_{end} for the end of measurement are determined according to the different lesion types and lesion lengths, reducing interference, increasing accuracy, and helping physicians to learn the exact length and position of lesions.

Optionally, for discontinuous serial lesions, the method of determining the measurement range for Computed Tomography Fractional Flow Reserve comprises:
calculating a centerline point MP_{beg} for the start of measurement and a centerline point MP_{end} for the end of measurement:

$${MP}_{beg} = {CLP}_{normal from {LP}_{dis}}$$

wherein the centerline point MP_{end} for the start of measurement is the position of the centerline point where normal blood vessel diameter is restored from the previous lesion;

$${MP}_{end} = {MP}_{beg} + ( 0.5 to 1 ) mm$$

wherein the centerline point MP_{end} for the end of measurement is 0.5 mm-1 mm longitudinally along the coronary artery from the centerline point MP_{beg} for the start of measurement in the lesion distal direction.

In this solution, the centerline point MP_{beg} for the start of measurement and the centerline point MP_{end} for the end of measurement are determined according to the different lesion types and lesion lengths, reducing interference, increasing accuracy, and helping physicians to learn the exact length and position of lesions.

Optionally, if a distal end of a lesion is close to the farthest end of the coronary artery, then a centerline point at a position where lumen diameter is 1.5 mm is taken to be the lesion distal centerline point when determining the focus length of the lesion position, and a centerline point at a position where lumen diameter is 1.5 mm is taken to be the centerline point MP_{end} for the end of measurement when determining the measurement range for Computed Tomography Fractional Flow Reserve:

$${MP}_{end} = 1.5 mm lumen diameter centerline point .$$

In this solution, a typical or average diameter of the distal end of a lesion is generally 1.5 mm, so choosing this point as a reference can help a physician to assess the length and severity of the lesion more accurately.

Optionally, the method of performing lesion specific classification comprises:

when the maximum value of all the CT-FFR values within the measurement range is less than or equal to a cut-off value 0.7, the mean value of all the CT-FFR values within the measurement range is used as the CT-FFR value of the lesion, and the lesion is labeled as positive;

when the minimum value of all the CT-FFR values within the measurement range is greater than or equal to a cut-off value 0.85, the mean value of all the CT-FFR values within the measurement range is used as the CT-FFR value of the lesion, and the lesion is labeled as negative;

when there are CT-FFR values within the measurement range that are distributed between 0.7 and 0.85, a histogram is established on the basis of all the CT-FFR values, a category with the highest frequency in the histogram is determined, and the mean value of CT-FFR values in this category is calculated and compared with the cut-off values; if the mean value is less than or equal to 0.7, the lesion is determined as being a positive lesion, and labeled as positive; if the mean value is greater than or equal to 0.85, the lesion is determined as being a negative lesion, and labeled as negative; if the mean value is greater than 0.7 but less than 0.85, the lesion is determined as being in a gray zone.

In this solution, lesion specific classification of lesions in a hemodynamical sense is performed by a histogram statistical method, displaying the distribution characteristics of CT-FFR values in a visually direct and effective way, thus enhancing the accuracy and stability of CT-FFR assessment, reducing interference, and providing a more reliable solution for clinical physicians and patients.

Another technical solution of the present disclosure provides a system for performing measurement and classification for Computed Tomography Fractional Flow Reserve, used to realize the method for performing measurement and classification for Computed Tomography Fractional Flow Reserve, the system comprising:
a lesion type classification module, for dividing lesions into different types, according to branch involvement and calcification and length of lesions displayed in a Coronary Computed Tomography Angiography (CCTA) image;
a focus length determining module for determining focus lengths of lesion positions according to the different lesion types;
a measurement range determining module for determining measurement ranges for Computed Tomography Fractional Flow Reserve according to the focus lengths of the different lesion types;
a CT-FFR value calculating module for calculating all Computed Tomography Fractional Flow Reserve (CT-FFR) values within the measurement ranges;
a statistical analysis module for subjecting the Computed Tomography Fractional Flow Reserve values to statistical analysis according to the measurement ranges, to obtain a lesion specific classification result.

In this solution, a lesion type classification function is provided, being able to identify lesions of different types, select a suitable focus length measurement strategy according to the different lesion types, and thereby determine measurement ranges for CT-FFR values of specific lesions, so as to increase the accuracy of measurement. By performing lesion specific classification of lesions in a hemodynamical sense, the accuracy and stability of CT-FFR assessment is enhanced, interference is reduced, and a more reliable solution is provided for clinical physicians and patients.

Another technical solution of the present disclosure provides a device for performing measurement and classification for Computed Tomography Fractional Flow Reserve, comprising: a memory, a processor, and a program stored on the memory and able to run on the processor, the program being configured to realize the method for performing measurement and classification for Computed Tomography Fractional Flow Reserve.

Another technical solution of the present disclosure provides a storage medium, wherein a program is stored on the storage medium, and the program, when executed by a processor, realizes the method for performing measurement and classification for Computed Tomography Fractional Flow Reserve.

In summary, the present disclosure provides a lesion type classification function, being able to identify lesions of different types such as single focal lesions, diffuse lesions and serial lesions, select a suitable focus length measurement strategy according to the different lesion types, and thereby determine measurement ranges for CT-FFR values of specific lesions, so as to increase the accuracy of measurement. By performing lesion specific classification of lesions in a hemodynamical sense, the accuracy and stability of CT-FFR assessment is enhanced, interference is reduced, and a more reliable solution is provided for clinical physicians and patients.

### BRIEF DESCRIPTION OF THE DRAWINGS

FIG. 1 illustrates a flow chart of an example method provided in an embodiment of the present disclosure for performing measurement and classification for CT-FFR;

FIG. 2 illustrates an example schematic diagram showing the determination of focus lengths and measurement ranges of a single focal lesion, a diffuse lesion and a continuous serial lesion;

FIG. 3 illustrates an example schematic diagram showing the determination of focus length and measurement range of a discontinuous serial lesion; and

FIG. 4 illustrates a block diagram of an example system provided in an embodiment of the present disclosure for performing measurement and classification for CT-FFR.

Reference numerals used in the drawings:
S1-S4: steps of a method provided in an embodiment for performing measurement and classification for CT-FFR;
LPₚᵣₒ: lesion proximal centerline point of entire lesion;
LP_{dis}: lesion distal centerline point of entire lesion;
LPₚᵣₒ′: lesion proximal centerline point of each lesion in a discontinuous serial lesion;
LP_{dis}′: lesion distal centerline point of each lesion in a discontinuous serial lesion;
MP_{beg}: centerline point for start of measurement;
MP_{end}: centerline point for end of measurement;
401: lesion type classification module;
402: focus length determining module;
403: measurement range determining module;
404: CT-FFR value calculating module;
405: statistical analysis module.

### DETAILED DESCRIPTION

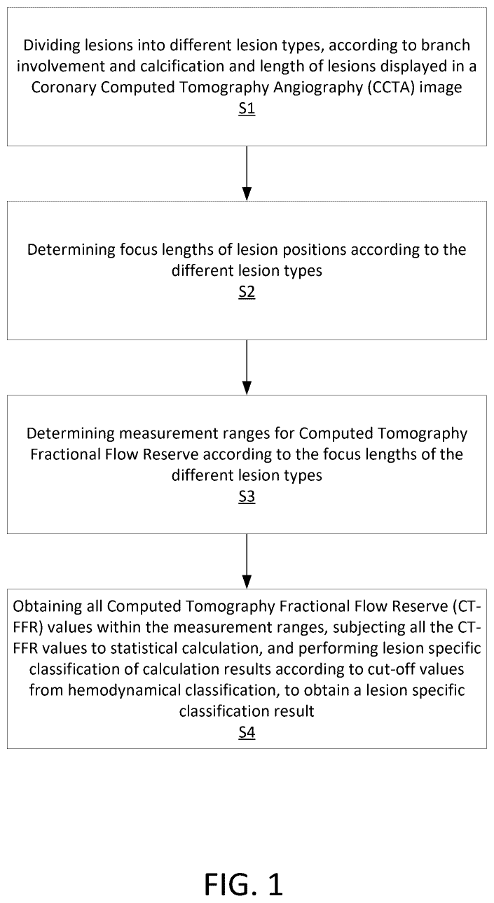

Embodiments of the present disclosure are explained in detail below with reference to FIGS. 1-4.

Fractional Flow Reserve (FFR) is an important reference standard for clinical evaluation of functional stenosis in coronary artery disease (CAD). FFR is calculated by measuring the ratio of distal coronary artery pressure (Pd) to aortic pressure (Pa) in a state of maximum coronary artery congestion, and has a value in the range of 0.0 to 1.0. The measurement of FFR is typically performed under the guidance of Percutaneous Coronary Intervention (PCI), and it is necessary to use a pressure guide wire and to ensure that a pressure sensor is positioned at least 2 cm-3 cm distal to the stenosis under assessment. It is generally considered that lesions with an FFR value≤0.8 will cause lesion-specific ischemia, while lesions with an FFR value>0.8 have little association with myocardial ischemia. When the FFR value is between 0.75 and 0.8, it is considered to be in the “gray zone”, and is of little diagnostic value.

When measuring FFR, it is necessary to closely monitor blood pressure and congestion in the patient; measurement is carried out when the lowest point level of the Pd/Pa trace has remained stable for 20 seconds after pressure system calibration, balancing of the distal coronary artery pressure Pd and aortic pressure Pa, and injection of adenosine. In the case of multiple continuous stenosis or diffuse atherosclerosis disease, it is necessary to slowly pull back the pressure sensor in a stable congestion state to assess the distribution of abnormal epicardial resistance, and examine the entire length of the artery within a time period of approximately 15-20 seconds.

Computed Tomography Fractional Flow Reserve (CT-FFR) is a non-invasive assessment technique, which is based on Coronary Computed Tomography Angiography (CCTA) data combined with an advanced computational analysis method such as computational fluid dynamics (CFD) or machine learning (ML). CT-FFR aims to simulate the physiological state of the coronary artery, providing simulated values similar to those of invasive FFR in order to assess the hemodynamical significance of coronary artery lesions. CT-FFR has good correlation with invasive FFR in terms of diagnostic performance and, compared with CCTA alone, shows potential with regard to optimizing patient management, improving risk stratification and prognosis.

The computation process of CT-FFR includes: using CCTA data to reconstruct an anatomical model of the coronary arteries, extracting coronary artery centerlines and lumen contour lines, then inputting the extracted coronary artery centerline tree, performing corresponding lumen segmentation at each centerline point, and using a machine learning algorithm to predict CT-FFR values of each centerline point along the centerline tree, to finally generate a color coded grid to display the CT-FFR values corresponding to each centerline point.

The CT-FFR measurement position is generally recommended to be 2 cm-3 cm distal to the stenosis, so as to have better correlation with invasive FFR. However, since the CT-FFR measurement position is chosen manually, and CT-FFR values within the range of 2 cm-3 cm distal to the stenosis might vary due to differences in centerline points and lumen contour, this might cause uncertainty or non-uniformity of measurement; e.g. when the CT-FFR values are in the gray zone, it may even cause misjudgments.

When a software algorithm is applied to perform automated detection and measurement, a fixed centerline point (e.g. a position 2 cm distal to the lesion) will generally be chosen as the measurement point for CT-FFR values. Although such a measurement method is simple, convenient, and quick in operation, and capable of obtaining a reference value rapidly, measurement at a fixed center point might neglect minute changes in actual blood flow in the lesion area, and a fixed center point alone might be insufficient to accurately reflect CT-FFR values for the entire lesion area and unable to fully take into account the complexity and diversity of lesions, so the precision and accuracy of measurement is reduced. Therefore, when choosing a measurement position for CT-FFR, lesions of different types should be taken into account together, and the measurement position should be chosen as precisely as possible, to ensure clinical relevance and accuracy of the measurement result.

In clinical practice, there are clear guiding principles for the selection of CT-FFR measurement position based on lesion type and distribution: for a single focal lesion, the measurement point is typically chosen manually 2 cm-3 cm distal to the stenosis; for a diffuse lesion of a length greater than or equal to 3 cm, or a continuous serial lesion with a spacing of less than or equal to 1 cm, measurement is performed 2 cm distal to the lesion; for an intermittent serial lesion with a spacing greater than or equal to 1 cm, it is recommended that measurement be performed at the distal end of each lesion. For the majority of stenosis lesions, the measurement points of distal epigenetically normal coronary arteries should extend from the distal end of the stenosis to 2 cm from the entire lesion.

On this basis, the present disclosure provides a method for performing automatic measurement and classification for CT-FFR; as shown in FIG. 1, the method comprises the following steps:
Step S1, dividing lesions into different lesion types, according to branch involvement and calcification and length of lesions displayed in a Coronary Computed Tomography Angiography (CCTA) image.
Step S2, determining focus lengths of lesion positions according to the different lesion types.
Step S3, determining measurement ranges for Computed Tomography Fractional Flow Reserve according to the focus lengths of the different lesion types.
Step S4, obtaining all Computed Tomography Fractional Flow Reserve (CT-FFR) values within the measurement ranges, subjecting all the CT-FFR values to statistical calculation, and performing lesion specific classification of calculation results according to cut-off values from hemodynamical classification, to obtain a lesion specific classification result.

The present disclosure provides a lesion type classification function being able to identify lesions of different types, select a suitable focus length measurement strategy according to the different lesion types, and thereby customize measurement ranges for CT-FFR values of specific lesions, so as to increase the accuracy of measurement. By performing lesion specific classification of lesions in a hemodynamical sense, the accuracy and stability of CT-FFR assessment is enhanced, interference is reduced, and a more reliable solution is provided for clinical physicians and patients.

In step S1, the lesion types include: single focal lesions, diffuse lesions, and serial lesions, wherein serial lesions are further divided into continuous serial lesions and discontinuous serial lesions.

In step S1, the method of dividing lesion types comprises:
measuring lesion lengths in the CCTA image, and analyzing branch involvement and degree of calcification of lesions in the CCTA image.

The Agatston score is commonly used clinically to assess the severity of calcification of the coronary arteries; generally, scores of 0, 1-100, 101-400, and more than 400 for calcification of the coronary arteries respectively correspond to four categories: no calcification, slight calcification, moderate calcification, and severe calcification.

Single focal lesions: if the length of a lesion is less than or equal to 10 mm, with mild or no calcification, and no principal branch involvement, then it is determined as being a single focal lesion. Single focal lesions also have other characteristics, such as incomplete occlusion (meaning that the blood vessel has not been blocked completely, and there is still some blood flowing through it), the position is not at an opening (the lesion is not located at an opening of the blood vessel, i.e. a starting region of a blood vessel branch), there is no thrombus (there is no thrombosis in the blood vessel), and the contour is smooth. According to the classification of coronary artery lesions by the American College of Cardiology (ACC) and the American Heart Association (AHA), single focal lesions are generally classified as typical type A lesions (simple lesions).

Diffuse lesions: if the length of a lesion is greater than or equal to 20 mm, with principal branch involvement, and excessive proximal twisting (meaning that abnormal bending or twisting has occurred in a proximal part of the blood vessel, i.e. a part nearer to the heart), then it is determined as being a diffuse lesion. According to the classification of coronary artery lesions by the American College of Cardiology (ACC) and the American Heart Association (AHA), diffuse lesions are generally classified as typical type C lesions (complex lesions).

Serial lesions: if the length of an entire lesion is more than 10 mm but less than 20 mm, with moderate or severe calcification, and an irregular contour, then it is determined as being a serial lesion; further, if lesions are spaced apart by less than 10 mm, then they are determined as being a continuous serial lesion, and if lesions are spaced apart by 10 mm or more, then they are determined as being a discontinuous serial lesion. According to the classification of coronary artery lesions by the American College of Cardiology (ACC) and the American Heart Association (AHA), serial lesions are generally classified as typical type B lesions (lesions of medium complexity).

By identifying lesions of different types such as single focal lesions, diffuse lesions and serial lesions, a suitable focus length measurement strategy can be chosen according to different lesion types, and measurement ranges for CT-FFR values of specific lesions can be determined automatically, so as to increase the accuracy of measurement.

In step S2, the method of determining focus lengths of lesion positions comprises:

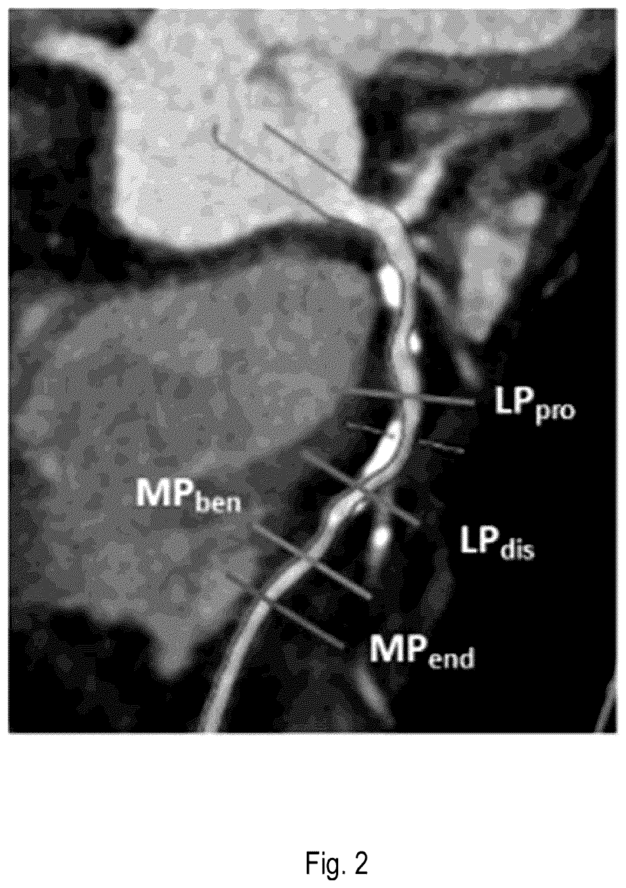

As shown in FIG. 2 for a single focal lesion, a lesion proximal centerline point LPₚᵣₒ and a lesion distal centerline point LP_{dis} are separately recorded; if the lesion is close to the farthest end of the coronary artery, then a centerline point at a position where the lumen diameter is 1.5 mm is taken to be the lesion distal centerline point LP_{dis}. A typical or average diameter of the distal end of a lesion is generally 1.5 mm, so choosing this point as a reference can help a physician to assess the length and severity of the lesion more accurately.

As shown in FIG. 2 for a diffuse lesion, a lesion proximal centerline point LPₚᵣₒ and a lesion distal centerline point LP_{dis} are separately recorded; if the lesion is close to the farthest end of the coronary artery, then a centerline point at a position where the lumen diameter is 1.5 mm can be taken to be the lesion distal centerline point LP_{dis}.

For a serial lesion LPₚᵣₒ and LP_{dis} of the entire lesion are first determined, then determination of continuity is performed, comprising: if there is a sudden change in the rate of change of radius in a middle part of the lesion, i.e. the diameter is greater than at the proximal end of the lesion, and the length is greater than 1 cm, then it is classified as a discontinuous serial lesion.

As shown in FIG. 2, for a continuous serial lesion, a lesion proximal centerline point LPₚᵣₒ and a lesion distal centerline point LP_{dis} are separately recorded; if the lesion is close to the farthest end of the coronary artery, then a centerline point at a position where the lumen diameter is 1.5 mm can be taken to be the lesion distal centerline point LP_{dis}

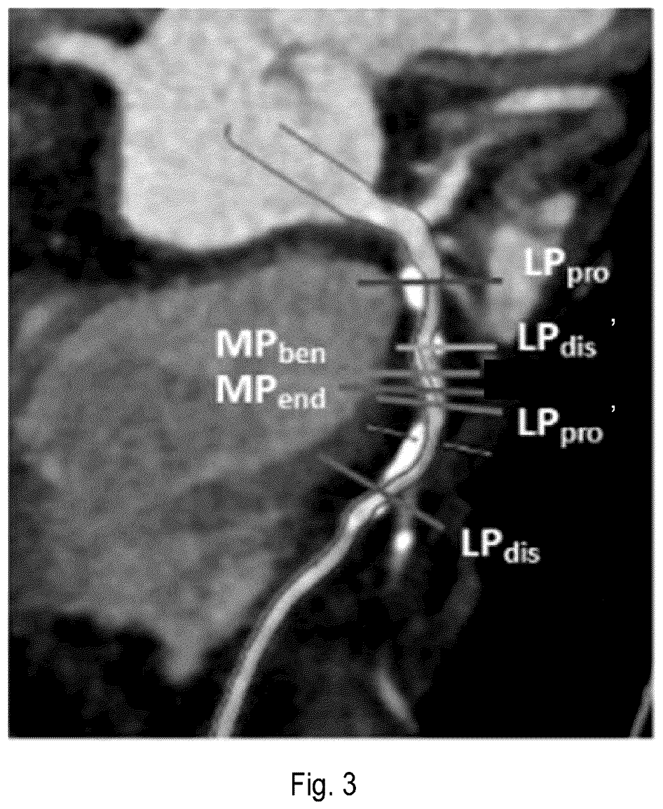

As shown in FIG. 3, for a discontinuous serial lesion, a lesion proximal centerline point LPₚᵣₒ and a lesion distal centerline point LP_{dis} of the entire lesion are recorded first, then a lesion proximal centerline point LPₚᵣₒ′ and a lesion distal centerline point LP_{dis}′ of each lesion are separately recorded. If the lesion is close to the farthest end of the coronary artery, then a centerline point at a position where the lumen diameter is 1.5 mm can be taken to be the lesion distal centerline point.

Based on the different lesion types, a matching method is chosen to determine the lesion proximal centerline point and lesion distal centerline point, so as to determine measurement ranges for CT-FFR values of specific lesions according to different focus lengths, in order to increase measurement accuracy.

In step S3, the method of determining measurement ranges for Computed Tomography Fractional Flow Reserve comprises:

As shown in FIG. 2, for single focus lesions, diffuse lesions, and continuous serial lesions, a centerline point MP_{beg} for the start of measurement and a centerline point MP_{end} for the end of measurement are calculated:

$${MP}_{beg} = {LP}_{dis} + 2 cm \qquad ( 1 )$$

wherein the centerline point MP_{beg} for the start of measurement is 2 cm longitudinally along the coronary artery from the lesion distal centerline point LP_{dis} in the lesion distal direction.

$${MP}_{end} = {LP}_{dis} + 3 cm \qquad ( 2 )$$

wherein the centerline point MP_{end} for the end of measurement is 3 cm longitudinally along the coronary artery from the lesion distal centerline point LP_{dis} in the lesion distal direction.

Determining the centerline point MP_{beg} for the start of measurement and the centerline point MP_{end} for the end of measurement helps the physician to learn the exact length and position of the lesion.

Alternatively:

If the distal end of the lesion is close to the farthest end of the coronary artery, then a

centerline point at a position where the lumen diameter is 1.5 mm is taken to be the centerline point MP_{end} for the end of measurement,
MP_{end}=1.5 mm lumen diameter centerline point (3);
choosing this point as a reference can help the physician to assess the length and severity of the lesion more accurately.

As shown in FIG. 3, for a discontinuous serial lesion, the centerline point MP_{beg} for the start of measurement and the centerline point MP_{end} for the end of measurement are calculated:

$${MP}_{beg} = {CLP}_{normal from {LP}_{dis}} \qquad ( 4 )$$

wherein the centerline point MP_{beg} for the start of measurement is the position of the centerline point where normal blood vessel diameter is restored from the previous lesion;

$${MP}_{end} = {MP}_{beg} + ( 0.5 to 1 ) mm \qquad ( 5 )$$

wherein the centerline point MP_{end} for the end of measurement is 0.5 mm-1 mm longitudinally along the coronary artery from the centerline point MP_{beg} for the start of measurement in the lesion distal direction.

Determining the centerline point MP_{beg} for the start of measurement and the centerline point MP_{end} for the end of measurement helps the physician to learn the exact length and position of the lesion.

Alternatively, if the distal end of the lesion is close to the farthest end of the coronary artery, then a centerline point at a position where the lumen diameter is 1.5 mm is taken to be the centerline point MP_{end} for the end of measurement, MP_{end}=1.5 mm lumen diameter centerline point (6); choosing this point as a reference can help the physician to assess the length and severity of the lesion more accurately.

In step S4, the CT-FFR values are statistically analyzed according to the measurement ranges, to obtain a lesion specific classification result.

All Computed Tomography Fractional Flow Reserve (CT-FFR) values within the measurement ranges are obtained, statistical matrices are established to subject all the values to calculation, and the calculation results are classified according to cut-off values from hemodynamical classification. The CT-FFR sampling interval and sampling frequency are generally related to CT scan parameter settings, and the specific values might vary for different CT apparatuses and scanning protocols.

The statistical matrix includes: a minimum value, a maximum value, a mean value, and a histogram.

The cut-off values are 0.7 and 0.85; if the CT-FFR value≤0.7, the lesion is determined as being a positive lesion; if the CT-FFR value≥0.85, the lesion is determined as being a negative lesion; if the CT-FFR value is between these two values, the lesion will be determined as being in the gray zone, signifying that further clinical evidence is needed to perform classification.

When the maximum value of all the CT-FFR values within the measurement range is less than or equal to 0.7, the mean value of all the CT-FFR values within the measurement range is used as the CT-FFR value of the lesion, and the lesion is labeled as positive, e.g. labeled red.

When the minimum value of all the CT-FFR values within the measurement range is greater than or equal to 0.85, the mean value of all the CT-FFR values within the measurement range is used as the CT-FFR value of the lesion, and the lesion is labeled as negative, e.g. labeled blue.

When there are CT-FFR values within the measurement range that are distributed between 0.7 and 0.85, a histogram is established on the basis of all the CT-FFR values, a category with the highest frequency in the histogram is determined, and the mean value of CT-FFR values in this category is calculated and compared with the cut-off values; if the mean value is less than or equal to 0.7, the lesion is determined as being a positive lesion, and labeled as positive; if the mean value is greater than or equal to 0.85, the lesion is determined as being a negative lesion, and labeled as negative; if the mean value is greater than 0.7 but less than 0.85, the lesion is determined as being in the gray zone, and labeled as being in the gray zone, e.g. labeled gray.

The use of a histogram to analyze the CT-FFR values provides a visually direct and effective means of visualizing data, which is not only able to clearly display distribution characteristics of the CT-FFR values, including concentration trends, degree of dispersion and data density, but also helps to quickly identify abnormal values and outlying points. Histograms enable physicians to compare CT-FFR value distributions of different patient groups, to assist with diagnostic decisions, and judge whether data is within a normal range. The adjustability of histograms allows physicians to adjust group numbers according to different analysis requirements, to obtain more precise or more general views of data, so as to better assess and interpret CT-FFR data.

The present disclosure divides lesions into different types according to a Coronary Computed Tomography Angiography (CCTA) image, determines focus lengths of lesion positions according to different lesion types, then determines Computed Tomography Fractional Flow Reserve measurement ranges according to the focus lengths of different lesion types, establishes statistical matrices to subject all Computed Tomography Fractional Flow Reserve (CT-FFR) values within the measurement ranges to calculation, and finally subjects the calculation results to hemodynamical classification.

The present disclosure introduces a lesion type classification function, being able to automatically identify lesions of different types such as single focal lesions, diffuse lesions, and serial lesions, select a suitable focus length measurement strategy according to the different lesion types, and thereby determine measurement ranges for CT-FFR values of specific lesions, so as to increase measurement accuracy. The present disclosure performs lesion specific classification of lesions in a hemodynamical sense, enhancing the accuracy and stability of CT-FFR assessment, reducing interference, and providing a more reliable solution for clinical physicians and patients.

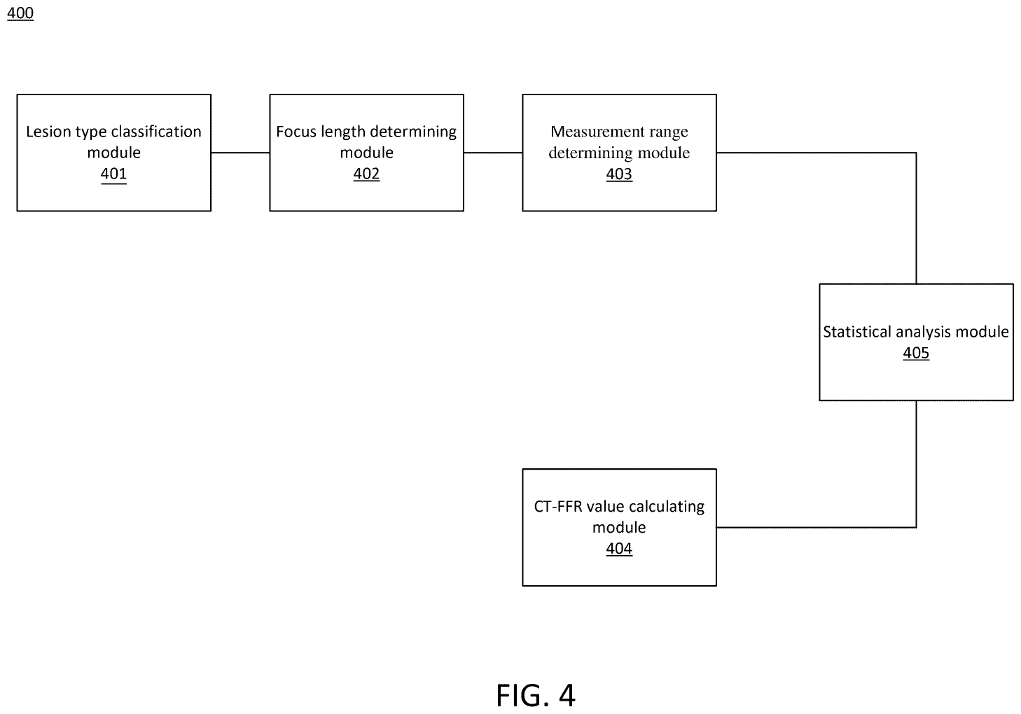

As shown in FIG. 4, the present disclosure further provides a system for performing measurement and classification for Computed Tomography Fractional Flow Reserve, comprising:
a lesion type classification module 401, configured to divide lesions into different types, according to branch involvement and calcification and length of lesions displayed in a Coronary Computed Tomography Angiography (CCTA) image;
a focus length determining module 402, configured to determine focus lengths of lesion positions according to the different lesion types;
a measurement range determining module 403, configured to determine measurement ranges for Computed Tomography Fractional Flow Reserve according to the focus lengths of the different lesion types;
a CT-FFR value calculating module 404, configured to calculate all Computed Tomography Fractional Flow Reserve (CT-FFR) values within the measurement ranges;
a statistical analysis module 405, configured to subject the Computed Tomography Fractional Flow Reserve values to statistical analysis according to the measurement ranges, to obtain a lesion specific classification result.

The present disclosure introduces a lesion type classification function, being able to identify lesions of different types such as single focal lesions, diffuse lesions and serial lesions, select a suitable focus length measurement strategy according to the different lesion types, and thereby determine measurement ranges for CT-FFR values of specific lesions, so as to increase measurement accuracy. The present disclosure performs lesion specific classification of lesions in a hemodynamical sense, enhancing the accuracy and stability of CT-FFR assessment, reducing interference, and providing a more reliable solution for clinical physicians and patients.

The present disclosure further provides an apparatus for performing measurement and classification for Computed Tomography Fractional Flow Reserve, comprising: a memory, a processor, and a program stored on the memory and able to run on the processor, the program being configured to realize the method for performing measurement and classification for Computed Tomography Fractional Flow Reserve.

The present disclosure further provides a storage medium, the storage medium having a program stored thereon, wherein the program, when executed by the processor, realizes the method for performing measurement and classification for Computed Tomography Fractional Flow Reserve.

The present disclosure introduces a lesion type classification function, being able to identify lesions of different types such as single focal lesions, diffuse lesions and serial lesions, select a suitable focus length measurement strategy according to the different lesion types, and thereby determine measurement ranges for CT-FFR values of specific lesions, so as to increase measurement accuracy. The present disclosure performs lesion specific classification of lesions in a hemodynamical sense, enhancing the accuracy and stability of CT-FFR assessment, reducing interference, and providing a more reliable solution for clinical physicians and patients.

It should be explained that in embodiments of the present disclosure, orientations or positional relationships indicated by terms such as “central”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “anticlockwise”, “axial”, “radial” and “circumferential” are based on the orientations or positional relationships shown in the drawings, and are only intended to facilitate the description of embodiments, without indicating or implying that the device or element referred to must have a specific orientation or be constructed and operated in a specific orientation, and therefore must not be construed as limiting the present disclosure. In addition, the terms “first”, “second” and “third” are merely used to describe objects, and must not be construed as indicating or implying relative importance.

In the present disclosure, unless otherwise clearly specified and defined, terms such as “install”, “connected”, “connect” and “fix” should be understood in a broad sense, e.g. may mean a fixed connection, a detachable connection or integral forming; a mechanical connection or an electrical connection; a direct connection, an indirect connection via an intermediate medium, an internal connection of two elements, or an interactive relationship between two elements. Those skilled in the art will be able to understand the specific meanings of the abovementioned terms in the present disclosure according to the specific circumstances.

It will be understood that as used in this description and the appended claims, the term “comprises” indicates the presence of the described feature, entity, step, operation, element and/or assembly, but does not rule out the presence or addition of one or more other feature, entity, step, operation, element, assembly and/or set thereof.

It should also be understood that terms used in the description of the present application herein are for the sole purpose of describing specific embodiments, and are not intended to limit the present application. As used in the description of the present application and the appended claims, “a”, “an”, and “the” in the singular form are intended to include the plural form, unless other circumstances are clearly indicated in the context.

It should be further understood that the term “and/or” used in the description of the present application and the appended claims refers to any combination and all possible combinations of one or more of the associated listed items, and includes these combinations.

As used in this description and the appended claims, the term “if” may, depending on the context, be interpreted as “when . . . ” or “once” or “in response to determining” or “in response to detecting”. Similarly, the phrase “if it is determined” or “if [the described condition or event] is detected” may, depending on the context, be interpreted as meaning “once it is determined” or “in response to determining” or “once [the described condition or event] is detected” or “in response to detecting [the described condition or event]”.

Although the content of the present disclosure has already been presented in detail by means of the embodiments above, it should be recognized that the above description should not be regarded as a restriction of the present disclosure. Obviously, the embodiments described are merely some, not all, of the embodiments of the present disclosure. All other embodiments obtained by those skilled in the art on the basis of the embodiments in the present disclosure without any creative effort are included within the scope of protection of the present disclosure. Various amendments and substitutions for the present disclosure will be obvious to those skilled in the art after perusal of the content above. Thus, the scope of protection of the present disclosure shall be defined by the attached claims.

The various components described herein may be referred to as “modules.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, as applicable and relevant, may alternatively be referred to herein as “circuitry,” “controllers,” “processors,” or “processing circuitry,” or alternatively as noted herein.

What is claimed is:

1. A method for performing measurement and classification for Computed Tomography Fractional Flow Reserve (CT-FFR), comprising:
dividing lesions into different lesion types according to branch involvement, calcification, and length of lesions displayed in a Coronary Computed Tomography Angiography (CCTA) image;
determining focus lengths of lesion positions according to the different lesion types;
determining measurement ranges for CT-FFR according to the focus lengths of the different lesion types;
obtaining CT-FFR values within each of the measurement ranges;
subjecting the obtained CT-FFR values to statistical calculations; and
performing lesion specific classification of the results of the statistical calculations according to cut-off values from hemodynamical classification, to thereby obtain the lesion specific classification result.

2. The method as claimed in claim 1, wherein the dividing the lesions into different lesion types comprises:
when the length of a lesion is less than or equal to 10 mm, with calcification less than a threshold value and no principal branch involvement, then the lesion is determined as being a single focal lesion;
when the length of a lesion is greater than or equal to 20 mm, with principal branch involvement and proximal twisting exceeding a threshold value, then the lesion is determined as being a diffuse lesion;
when the length of a lesion is more than 10 mm but less than 20 mm, with calcification exceeding a threshold value and an irregular contour, then the lesion is determined as being a serial lesion;
when lesions are spaced apart by less than 10 mm, the lesions are determined as being a continuous serial lesion; and
when lesions are spaced apart by 10 mm or more, the lesions are determined as being a discontinuous serial lesion.

3. The method as claimed in claim 2, wherein the determining the focus lengths of lesion positions comprises:
for a single focal lesion, separately recording a lesion proximal centerline point LPₚᵣₒ and a lesion distal centerline point LP_{dis};
for a diffuse lesion, separately recording a lesion proximal centerline point LPₚᵣₒ and a lesion distal centerline point LP_{dis};
for a serial lesion, first determining a proximal centerline point LPₚᵣₒ and a lesion distal centerline point LP_{dis} of the serial lesion, and determining a continuity of the serial lesion to determine whether the serial lesion is a continuous serial lesion or a discontinuous serial lesion;
for a continuous serial lesion, separately recording a lesion proximal centerline point LPₚᵣₒ and a lesion distal centerline point LP_{dis}; and
for a discontinuous serial lesion, first recording a lesion proximal centerline point LPₚᵣₒ and a lesion distal centerline point LP_{dis} of the discontinuous serial lesion, then separately recording a lesion proximal centerline point LPₚᵣₒ′ and a lesion distal centerline point LP_{dis}′ of each lesion of the discontinuous serial lesion.

4. The method as claimed in claim 3, wherein the determining the measurement range for CT-FFR comprises:
(i) for single focal lesions, diffuse lesions, and continuous serial lesions:
calculating a centerline point MP_{beg} by evaluating MP_{beg}=LP_{dis}+2 cm for a start of measurement and a centerline point MP_{end} by evaluating MP_{end}=LP_{dis}+3 cm for an end of a respective measurement,
wherein the centerline point MP_{beg} for the start of measurement is 2 cm longitudinally along a coronary artery from a respective lesion distal centerline point LP_{dis} in the lesion distal direction, and
wherein the centerline point MP_{end} for the end of measurement is 3 cm longitudinally along a coronary artery from the lesion distal centerline point LP_{dis} in the lesion distal direction; or
(ii) for discontinuous serial lesions:
calculating a centerline point MP_{beg} by evaluating MP_{beg}=CLP normal from LP_{dis} for a start of measurement and a centerline point MP_{end} by evaluating MP_{end}=MP_{beg}+(0.5 to 1) mm for an end of measurement,
wherein the centerline point MP_{beg} for the start of measurement is the position of the centerline point where normal blood vessel diameter is restored from a previous lesion, and
wherein the centerline point MP_{end} for the end of measurement is 0.5 mm-1 mm longitudinally along a coronary artery from the centerline point MP_{beg} for the start of measurement in the lesion distal direction.

5. The method as claimed in claim 4, wherein when a distal end of a lesion is close to the farthest end of the coronary artery, a centerline point at a position where a lumen diameter is 1.5 mm is taken as the lesion distal centerline point when determining the respective focus length of the lesion position, and a centerline point at a position where a lumen diameter is 1.5 mm is taken as the centerline point MP_{end} for the end of measurement when determining the measurement range for the measurement range for CT-FFR by evaluating:
$${MP}_{end} = 1.5 mm lumen diameter centerline point .$$

6. The method as claimed in claim 4, wherein the performing the lesion specific classification comprises:
when a maximum value of CT-FFR values within a respective measurement range is less than or equal to a cut-off value 0.7, a mean value of the CT-FFR values within the respective measurement range is used as the CT-FFR value of the respective lesion and the lesion is labeled as positive;
when the minimum value of CT-FFR values within a respective measurement range is greater than or equal to a cut-off value 0.85, a mean value of the CT-FFR values within the respective measurement range is used as the CT-FFR value of the respective lesion and the lesion is labeled as negative; and
when there are CT-FFR values within a respective measurement range that are distributed between 0.7 and 0.85:
calculating a histogram based upon the respective CT-FFR values;
determining a category with a highest frequency in the histogram;
calculate a mean value of CT-FFR values in the determined category; and
compare the mean value of CT-FFR values in the determined category with the cut-off values of 0.7 and 0.85;
when a mean value is less than or equal to 0.7, determining and labeling the lesion as a positive lesion;
when a mean value is greater than or equal to 0.85, determining and labeling the lesion as a negative lesion; and
when the mean value is greater than 0.7 but less than 0.85, determining the lesion as indeterminate.

7. A device for performing measurement and classification for Computed Tomography Fractional Flow Reserve (CT-FFR), comprising:
a memory configured to store computer-readable instructions; and
a processor configured to execute the computer-readable instructions stored on the memory to:
divide lesions into different lesion types according to branch involvement, calcification, and length of lesions displayed in a Coronary Computed Tomography Angiography (CCTA) image;
determine focus lengths of lesion positions according to the different lesion types;
determine measurement ranges for CT-FFR according to the focus lengths of the different lesion types;
obtain CT-FFR values within each of the measurement ranges;
subject the obtained CT-FFR values to statistical calculations; and
perform lesion specific classification of the results of the statistical calculations according to cut-off values from hemodynamical classification, to thereby obtain the lesion specific classification result.

8. The device as claimed in claim 7, wherein the processor is configured to execute the computer-readable instructions stored on the memory to divide the lesions into different lesion types by determining:
when the length of a lesion is less than or equal to 10 mm, with calcification less than a threshold value and no principal branch involvement, then the lesion is determined as being a single focal lesion;
when the length of a lesion is greater than or equal to 20 mm, with principal branch involvement and proximal twisting exceeding a threshold value, then the lesion is determined as being a diffuse lesion;
when the length of a lesion is more than 10 mm but less than 20 mm, with calcification exceeding a threshold value and an irregular contour, then the lesion is determined as being a serial lesion;
when lesions are spaced apart by less than 10 mm, the lesions are determined as being a continuous serial lesion; and
when lesions are spaced apart by 10 mm or more, the lesions are determined as being a discontinuous serial lesion.

9. The device as claimed in claim 8, wherein the processor is configured to execute the computer-readable instructions stored on the memory to determine the focus lengths of lesion positions by:
for a single focal lesion, separately recording a lesion proximal centerline point LPₚᵣₒ and a lesion distal centerline point LP_{dis};
for a diffuse lesion, separately recording a lesion proximal centerline point LPₚᵣₒ and a lesion distal centerline point LP_{dis};
for a serial lesion, first determining a proximal centerline point LPₚᵣₒ and a lesion distal centerline point LP_{dis} of the serial lesion, and determining a continuity of the serial lesion to determine whether the serial lesion is a continuous serial lesion or a discontinuous serial lesion;
for a continuous serial lesion, separately recording a lesion proximal centerline point LPₚᵣₒ and a lesion distal centerline point LP_{dis}; and
for a discontinuous serial lesion, first recording a lesion proximal centerline point LPₚᵣₒ and a lesion distal centerline point LP_{dis} of the discontinuous serial lesion, then separately recording a lesion proximal centerline point LPₚᵣₒ′ and a lesion distal centerline point LP_{dis}′ of each lesion of the discontinuous serial lesion.

10. The device as claimed in claim 9, wherein the processor is configured to execute the computer-readable instructions stored on the memory to determine the measurement range for CT-FFR by:
(i) for single focal lesions, diffuse lesions, and continuous serial lesions:
calculating a centerline point MP_{beg} by evaluating MP_{beg}=LP_{dis}+2 cm for a start of measurement and a centerline point MP_{end} by evaluating MP_{end}=LP_{dis}+3 cm for an end of a respective measurement,
wherein the centerline point MP_{beg} for the start of measurement is 2 cm longitudinally along a coronary artery from a respective lesion distal centerline point LP_{dis} in the lesion distal direction, and
wherein the centerline point MP_{end} for the end of measurement is 3 cm longitudinally along a coronary artery from the lesion distal centerline point LP_{dis} in the lesion distal direction; or
(ii) for discontinuous serial lesions:
calculating a centerline point MP_{beg} by evaluating MP_{beg}=CLP_{normal from LP}_{_{dis}} for a start of measurement and a centerline point MP_{end} by evaluating MP_{end}=MP_{beg}+(0.5 to 1) mm for an end of measurement,
wherein the centerline point MP_{beg} for the start of measurement is the position of the centerline point where normal blood vessel diameter is restored from a previous lesion, and
wherein the centerline point MP_{end} for the end of measurement is 0.5 mm-1 mm longitudinally along a coronary artery from the centerline point MP_{beg} for the start of measurement in the lesion distal direction.

11. The device as claimed in claim 10, wherein when a distal end of a lesion is close to the farthest end of the coronary artery, a centerline point at a position where a lumen diameter is 1.5 mm is taken as the lesion distal centerline point when determining the respective focus length of the lesion position, and a centerline point at a position where a lumen diameter is 1.5 mm is taken as the centerline point MP_{end} for the end of measurement when determining the measurement range for the measurement range for CT-FFR by evaluating:
$${MP}_{end} = 1.5 mm lumen diameter centerline point .$$

12. The device as claimed in claim 10, wherein the lesion specific classification comprises:
when a maximum value of CT-FFR values within a respective measurement range is less than or equal to a cut-off value 0.7, a mean value of the CT-FFR values within the respective measurement range is used as the CT-FFR value of the lesion and the lesion is labeled as positive;
when the minimum value of CT-FFR values within a respective measurement range is greater than or equal to a cut-off value 0.85, a mean value of the CT-FFR values within the respective measurement range is used as the CT-FFR value of the lesion and the lesion is labeled as negative; and
when there are CT-FFR values within a respective measurement range that are distributed between 0.7 and 0.85:
calculating a histogram based upon the respective CT-FFR values;
determining a category with a highest frequency in the histogram;
calculate a mean value of CT-FFR values in the determined category; and
compare the mean value of CT-FFR values in the determined category with the cut-off values of 0.7 and 0.85;
when a mean value is less than or equal to 0.7, determining and labeling the lesion as a positive lesion;
when a mean value is greater than or equal to 0.85, determining and labeling the lesion as a negative lesion; and
when the mean value is greater than 0.7 but less than 0.85, determining the lesion as indeterminate.

13. A non-transitory computer-readable medium having instructions stored thereon that, when executed by a processor, causes the processor to perform measurement and classification for Computed Tomography Fractional Flow Reserve (CT-FFR), by:
dividing lesions into different lesion types according to branch involvement, calcification, and length of lesions displayed in a Coronary Computed Tomography Angiography (CCTA) image;
determining focus lengths of lesion positions according to the different lesion types;
determining measurement ranges for CT-FFR according to the focus lengths of the different lesion types;
obtaining CT-FFR values within each of the measurement ranges;
subjecting the obtained CT-FFR values to statistical calculations; and
performing lesion specific classification of the results of the statistical calculations according to cut-off values from hemodynamical classification, to thereby obtain the lesion specific classification result.
